(19) United States
(12) Patent Application Publication
(10) Pub. No.: US 2004/0147917 A1
(43) Pub. Date: Jul. 29, 2004

(54) Device and method for treatment of breast tissue with electromagnetic radiation

(76) Inventors: Richard L. Mueller JR., Jackson, WY (US); James Kermode, Los Altos, CA (US)

Correspondence Address: OLSON & HIERL, LTD., 36th Floor, 20 North Wacker Drive, Chicago, IL, 60606, US

(21) Appl. No.: 10/349,569

(22) Filed: Jan. 23, 2003

(51) Int. Cl. A61B 18/04

(52) U.S. Cl. 606/33

(57) ABSTRACT

A medical device suitable for delivering electromagnetic energy, such as radiofrequency (RF) or microwave energy, to breast tissue in need of thermal treatment includes a hollow catheter sized to fit within a mammary duct of a patient and defining at least one passageway having a distal end portion and an open proximal end. An elongate insulating sleeve is slidably disposed within the passageway. An electromagnetic energy transmission line is disposed within the sleeve. The energy transmission line terminates, at its distal end, in an elongate, flexible, ablation probe. The ablation probe is configured for generating an electromagnetic field sufficient to cause tissue ablation and the probe is adapted for penetration of breast tissue. In use the catheter is passed through the orifice of a mammary duct and positioned within the duct adjacent to a region of breast tissue in need of thermal treatment, such as cancerous or pre-cancerous breast tissue. The ablation probe is passed through the passageway of the catheter and into the breast tissue to be treated. Electromagnetic energy is supplied to the ablation probe, heating the breast tissue adjacent to the probe. The breast tissue is heated to a temperature sufficient to ablate or otherwise destroy cancerous or pre-cancerous tissue. In a preferred embodiment the ablation probe includes a removable auger adapted for collecting a biopsy specimen of the breast tissue prior to thermal treatment, subsequent to thermal treatment, or both.

### FIELD OF THE INVENTION

The invention generally relates to methods and devices for treating cancerous and precancerous breast tissue. More particularly the invention relates to devices and methods for delivery of electromagnetic energy to breast tissue.

### BACKGROUND

Breast cancer is one of the most common cancers in women. Breast cancer can be difficult to diagnose in its early stages, prior to development of palpable lumps in the breast tissue. When diagnosed, treatments can vary from irradiation and chemotherapy, to lumpectomy (i.e, removal of the tumor tissue), to mastectomy (i.e., removal of the entire breast), or any combination thereof. Mastectomy can be very effective in preventing metastasis of breast cancer to other areas of the body, but can have devastating psychological effects in some patients.

There is an ongoing need for breast cancer treatments that minimize surgical intervention. U.S. Pat. No. 6,391,026 to Hung et al. describe a catheter device designed to be introduced through the orifice of a breast nipple duct and positioned within a mammary duct that has a cancerous or precancerous region therein. The catheter contains an electrode for delivering thermal energy to the duct to destroy the cancerous or pre-cancerous lining of the duct and tissue immediately surrounding the lining. When introduced through a nipple duct orifice, the device is specifically designed for treating the duct lining and is not suitable for selectively treating breast tissue outside of the duct.

Other devices are designed to be inserted through the exterior surface of the breast by piercing the skin. Such treatments are painful and traumatic for the patient.

There is an ongoing need for relatively selective, non-invasive, non-surgical methods of thermally treating breast tissue and that can be introduced into the breast tissue through the orifice of a breast nipple. The medical devices of the present invention fulfill this need.

### SUMMARY OF THE INVENTION

A medical device suitable for delivering electromagnetic energy, such as radiofrequency (RF) or microwave energy, to breast tissue in need of thermal treatment includes a hollow catheter provided with an ablation probe and sized to fit within a mammary duct of a patient. The catheter defines at least one passageway having a distal end portion and an open proximal end. An elongate insulating sleeve is slidably disposed within the passageway. An electromagnetic energy transmission line is disposed within the sleeve. The energy transmission line terminates, at its distal end, in an ablation probe. The ablation probe preferably is hollow so that biopsy samples can be obtained prior to tissue irradiation and/or after irradiation, as desired. The ablation probe is configured for generating an electromagnetic field sufficient to cause tissue ablation and is adapted for penetration of breast tissue from the mammary duct. The ablation probe can be a microwave antenna or an electrode configured to deliver RF energy to breast tissue, depending on the source of electromagnetic energy.

In one preferred embodiment, a medical device suitable for delivering electromagnetic energy to breast tissue in need of thermal treatment includes a hollow catheter defining a passageway having a distal end portion and an open proximal end The distal end portion of the passageway can be open or closed. When closed, the distal end portion is provided with a deflector, and the catheter defines a side port adjacent to the deflector, creating an access opening from the passageway to the exterior of the catheter. An elongate insulating sleeve containing an electromagnetic energy transmission line is slidably disposed within the passageway. The proximal end of the transmission line is adapted for connection to an electromagnetic energy generator such as an RF generator or a microwave generator. The distal end of the transmission line terminates in an ablation probe such as an RF electrode or a microwave antenna. The ablation probe is configured for penetration of breast tissue from the interior of a mammary duct and delivery of electromagnetic energy (preferably RF energy) thereto. The catheter has an external cross-sectional dimension (diameter) sized to fit within the lumen of a human mammary duct.

In another preferred embodiment, the device includes a hollow catheter defining a probe passageway and a viewing passageway, each having a distal end portion and an open proximal end. The distal end portion of the probe passageway can be open or closed. When the distal end portion of the probe passageway is closed, an optional deflector can be provided within the distal end portion thereof, in which case the catheter also defines a side port adjacent to the deflector. An elongate insulating sleeve containing an electromagnetic energy transmission line is slidably disposed within the probe passageway, and preferably is moveable therein. The proximal end of the transmission line is adapted for connection to an electromagnetic energy generator such as an RF or microwave generator. The distal end of the transmission line terminates in a hollow ablation probe adapted for penetration of breast tissue and delivery of electromagnetic energy thereto. The viewing passageway has open distal and proximal ends. A fiber optic viewing scope is disposed within the viewing passageway. The viewing scope is adapted for transmitting images from the open distal end of the viewing passageway to an external viewing apparatus, such as a video monitor, and the like. The viewing scope allows the clinician to accurately position the device within the mammary duct of a patient prior to treatment.

Preferably, the transmission line and the ablation probe are both hollow and can receive therewithin a fiber optic viewing scope and/or a removable auger. The auger, if present, is adapted for penetration of breast tissue and facilitates the collection of a biopsy sample. Often, however, the hollow ablation probe itself can collect the biopsy sample and the auger can be utilized to retrieve the collected sample from the hollow probe. The auger can itself serve as the RF electrode when appropriately connected to the energy transmission line. The biopsy sample can also be retrieved for tissue analysis by screwing the auger into the breast tissue distal to the ablation probe, and then pulling the sample back into the ablation probe and out through hollow transmission line. An optional thermal sensor, such as a thermistor can be positioned at the tip of the probe to sense the probe temperature and/or the temperature of the surrounding tissue. Preferably the thermal sensor is insulated from the ablation probe by a plastic or rubber sheath. The thermal sensor can be used to monitor the temperature of the tissue, or can be used with a feed back loop to control the temperature of the tissue and/or probe by regulating energy input to the probe.

The devices of the present invention are useful for thermal ablation of human breast tissue in a patient. In a preferred method, a clinician passes the catheter through the orifice of a human mammary duct in the nipple and positions the distal end portion of the catheter is within the duct adjacent to a region of breast tissue in need of thermal treatment, such as cancerous or pre-cancerous breast tissue. A fiber optic viewing scope within the catheter or within the hollow ablation probe carried by the catheter is used to position the catheter within the duct. Once the catheter is correctly placed, the breast is manipulated by the clinician so that the tip of the probe points at the wall of the duct adjacent to the breast tissue to be treated. The clinician then urges the ablation probe out through the open distal end (or the side port, depending on the device used) of the catheter, through the wall of the mammary duct, and into the breast tissue to be treated. Electromagnetic energy from a generator is suppled to the ablation probe by the energy transmission line. When the ablation probe is an RF electrode, alternating electric current supplied to the electrode resistively heats the surrounding breast tissue. When the ablation probe is a microwave antenna, the tissue is heated directly by the microwave radiation emitted therefrom. Preferably the tissue is heated to a temperature of at least about 55° C. to necrose or ablate the tissue.

When RF energy is used, the ablation probe is an electrode and is operably connected to an RF generator by the transmission line. RF energy, at a power level in the range of about 1 Watt (W) to about 10 W is supplied to the electrode for about 1 to about 120 seconds, at a frequency in the range of about 100 to about 750 KHz. If the electrode is monopolar, a dispersive electrode, also operably connected to the RF generator, is placed in contact with the patient's skin or other tissue to complete the circuit. If the electrode is bipolar, the dispersive electrode is not needed. The radiofrequency energy heats the electrode and breast tissue adjacent to the electrode to a temperature sufficient to inhibit cell proliferation or preferably to ablate (e.g., destroy) cancerous or pre-cancerous tissue, for example.

Alternatively, when the distal end portion of the catheter is closed and contains a deflector adjacent to a side port in the catheter, the ablation probe can be passed out through the side port, guided by the deflector, and into the region of breast tissue to be treated. The deflector can cause the probe to penetrate the duct wall and enter the breast tissue at an acute angle from the longitudinal axis of the catheter. In such case, the clinician does not need to manipulate the breast to insert the probe into the tissue.

The devices of the present invention can be supplied in a kit that includes instructional materials describing how to use the device to thermally treat breast tissue and accessories such as optical scopes, augers, a selection of different ablation probes, a dispersive electrode, and the like, and combinations thereof.

### BRIEF DESCRIPTION OF THE DRAWINGS

In the Drawings,

FIG. 1 is a partial cross-sectional view of an embodiment of the medical device of the present invention shown positioned for thermal treatment of breast tissue;

FIG. 2 is a cut-away view, partly in section, of the distal end portion of the device of FIG. 1 showing a needle-shaped electrode within the passageway of the catheter;

FIG. 3 is a partial cross-sectional view of the distal end portion of an alternative embodiment of the device of the present invention showing an insulating sleeve and a hollow ablation probe within the passageway of the catheter;

FIG. 4 depicts the device of FIG. 3 with an insulating sheath and hollow, open-ended electrode extended out through a side port in the catheter, and a biopsy auger within the hollow electrode;

FIG. 5 depicts an alternative embodiment with an auger-shaped RF electrode extended out from the open distal end of the insulating sleeve;

FIG. 6 shows an enlarged detail of an alternative, dual-passageway catheter embodiment of the device of the present invention positioned within a human breast for thermal treatment of breast tissue;

FIG. 7 depicts an enlarged detail of a particularly preferred embodiment of the medical device of the present invention that includes a fiber optic viewing scope and a hollow ablation probe; and

FIG. 8 depicts a variant of the device of FIG. 7 but including a biopsy auger in place of the viewing scope.

### DETAILED DESCRIPTION OF PREFERRED EMBODIMENTS

A medical device suitable for delivering electromagnetic energy to breast tissue in need of thermal treatment includes a hollow catheter sized to fit within a mammary duct of a patient and defining at least one passageway having a distal end portion and an open proximal end. An elongate insulating sleeve is slidably disposed within the passageway. An electromagnetic energy transmission line is disposed within the sleeve and terminates, at its distal end, in an elongate, flexible, ablation probe. The ablation probe is adapted for penetration of breast tissue from a mammary duct and when connected to an electromagnetic energy source, generates an electromagnetic field sufficient for tissue ablation.

In one preferred embodiment, the medical device includes a hollow catheter defining at least one passageway having an open distal end portion, an open proximal end. An elongate insulating sleeve containing an electromagnetic energy transmission line is slidably disposed within the passageway. The proximal end of the transmission line is adapted for connection to an electromagnetic energy generator, and the distal end of the transmission line terminates in an ablation probe. The ablation probe is adapted for penetration of breast tissue and delivery of electromagnetic energy thereto.

In another preferred embodiment the medical device includes a hollow catheter defining at least one passageway having a closed distal end portion, an open proximal end, and a side port adjacent to the distal end portion. The distal end portion of the passageway is provided with a deflector adjacent to the side port. An elongate insulating sleeve containing an electromagnetic energy transmission line operably connected to an ablation probe is slidably disposed within the passageway. The proximal end of the transmission line is adapted for connection to a electromagnetic energy generator, and the ablation probe at the distal end of the transmission line terminates is adapted for penetration of breast tissue and configured to generate an electromagnetic field within the tissue sufficient to necrose tissue that surrounds the probe.

In yet another preferred embodiment, the device includes a hollow catheter having at least two side-by-side passageways: a probe passageway having a distal end portion and an open proximal end, and a viewing passageway also having an open distal end and an open proximal end. An elongate insulating sleeve containing an elongate electromagnetic energy transmission line and an ablation probe is slidably disposed within the probe passageway. The ablation probe is adapted for penetrating breast tissue from the interior of a mammary duct and configured to generate an electromagnetic filed within the tissue sufficient to necrose or ablate the tissue adjacent to the probe. The proximal end of the transmission line is adapted for connection to an electromagnetic energy generator. The viewing passageway preferably contains a fiber optic viewing scope adapted for transmitting images from the open distal end of the viewing passageway to an external viewing apparatus, such as a video monitor, and the like.

Alternatively, the catheter can define a side port adjacent to the distal end portion of the probe passageway, and the distal end portion of the electrode passageway can be closed by a deflector for the ablation probe. The ablation probe can be urged through the passageway and out of the catheter through the side port, guided by the deflector. When the distal end of the catheter is positioned within a mammary duct the ablation probe can penetrate the wall of the duct and into the adjacent breast tissue as it exits the side port.

In particularly preferred embodiments the ablation probe is a hollow, open-ended RF electrode and the energy transmission line is also hollow, and in open communication with the electrode. The RF electrode and the transmission line together define a working passageway that can house a fiber optic viewing scope and/or a removable auger adapted for collection of breast tissue samples.

The catheter is sized to fit within the lumen of a human mammary duct. The catheter preferably has a diameter of no more than about 1.2 millimeters (i.e., about 4 French or smaller). The catheter can be made of any physiologically tolerable metal, such as stainless steel, or a polymeric (e.g., plastic) material, such as polyamide or polyurethane, as is well known in the art. Preferably the catheter is a stainless steel catheter.

The energy transmission line can be slidably disposed within the insulating sleeve so that the working electrode at the distal end of the conductor can be pulled within the insulator or extended outward therefrom. Preferably, the insulating sleeve is an insulating polymeric coating, such as a polyamide coating on the outer surface of the energy transmission line. The insulating sleeve provides thermal and electrical insulation for the electromagnetic transmission line. Preferably the insulating sleeve is made of a thermally-stable, insulating polymeric material such as a polyimide, a polyamide, a poly(tetrafluoroethylene) (e.g., TEFLON®), a silicone polymer, and the like. Preferably the sleeve has a wall thickness in the range of about 0.125 mm to about 0.05 mm, more preferably about 0.025 mm (i.e., about 1 mil).

The ablation probe is adapted for penetrating breast tissue through the interior wall of a mammary duct. For example, the ablation probe can be in the form of a needle, an auger, a screw, and the like. Preferably, the ablation probe is a microwave antenna, more preferably an RF electrode.

Probes that are microwave antennae are well known in the art. Suitable such devices are described in U.S. Pat. Nos. 6,471,696 and 6,325,796, both to Berube et al., and in U.S. Pat. No. 5,683,382 to Lenihan et al., the relevant disclosures of which are incorporated herein by reference.

RF electrodes suitable a ablation probes in the devices of the present invention are also well known in the art. The electrode is preferably monopolar, in which case it is used in conjunction with a dispersive electrode that is in electrical contact with the patient's body. Alternatively, the electrode can be bipolar. Bipolar electrodes do no utilize a dispersive electrode.

A particularly preferred ablation probe is an electrode in the form of a hollow coring needle, optionally having a biopsy auger situated therewithin. In this manner, a biopsy specimen of the breast tissue to be subjected to thermal treatment can be retrieved and analyzed prior to such treatment and after treatment, if desired. Optionally, the electrode itself can be in the form of a cork screw or auger and can retrieve a biopsy sample.

The electromagnetic energy transmission line preferably is a metal wire or a hollow, metal cannula. Suitable metals include stainless steel, nickel titanium alloy, and the like. The ablation probe can be integral with the energy transmission line, for example an uninsulated portion of the energy transmission line, or can be a separate component operably connected to the electrode.

The present device can be provided with a handle for facilitating manipulation of the catheter into position within a mammary duct. The handle can be provided with a mechanism for actuating transmittal of electromagnetic energy to the ablation probe, for manipulating the insulating sleeve, a fiber optic viewing scope, and/or the ablation probe through the passageways of the catheter, and the like.

The proximal end of the energy transmission line is adapted for connection to an electromagnetic energy generator such as a microwave or RF generator. Microwave and RF generators are well known in the art. Microwave generators suitable for medical use usually produce an alternating current having a frequency of about 915, 2450, or 2700 MHz. RF generators preferably produce alternating current having a frequency in the range of about 100 to about 750 KHz, more preferably about 500 to about 750 KHz.

In use, the distal end of the device is introduced through the orifice of a human mammary duct (i.e. through the orifice of a duct of the nipple) and is positioned within the lumen of the mammary duct adjacent to a suspect region of breast tissue in need of thermal treatment, such as cancerous or pre-cancerous tissue. The ablation probe is passed through a passageway of the catheter and out through the open distal end or side port, as the case may be. The probe penetrates the wall of the mammary duct and into the region of suspect breast tissue adjacent to the distal end of the passageway. The proximal end of the energy transmission line is operably connected to an electromagnetic energy generator such as an RF generator. When the ablation probe is a monopolar RF electrode, a dispersive electrode is also operably connected to the RF generator and is placed in electrical contact with the patients's body. The dispersive electrode preferably is in electrical contact with the external surface of the breast, or can be contacted subcutaneously.

When the RF generator is activated, the conductor preferably transmits about 1 to about 10 W of radiofrequency energy to the electrode at a frequency in the range of about 500 KHz to about 750 KHz. Preferably the energy is supplied in an amount and over a time period sufficient to heat the tissue to a temperature sufficient to prevent proliferation of the suspect breast tissue cells. Most preferably the suspect breast tissue cells are denatured, destroyed by the increased temperature of the tissue. At the same time, it is desirable to prevent thermal damage to healthy tissue in the mammary duct itself, or near the region of suspect tissue. The amount of energy applied and the duration of the treatment control the volume of tissue affected.

Alternating current from the generator heats the electrode, which in turn, heats the surrounding tissue to a temperature to at least about 40° C., more preferably about 55 to about 100° C., in order to necrose the tissue. The insulating sleeve and the catheter passageway provide thermal insulation to the mammary duct, thus preventing undesirable damage to the duct. Energy can be supplied to the probe for a few seconds to a few minutes, depending on the power level and frequency of the energy. Preferably the energy is supplied to the probe until the temperature of the surrounding tissue is at least about 55° C.

The distal end portion of the device can be provided with a thermal sensor, such as a thermistor or thermocouple, coupled with a temperature indicating device, such as a thermocouple meter, and the like, for monitoring the temperature of the tissue being heated. Alternatively, the thermal sensor can be used to control the temperature of the probe and/or tissue automatically through a feedback loop to the generator.

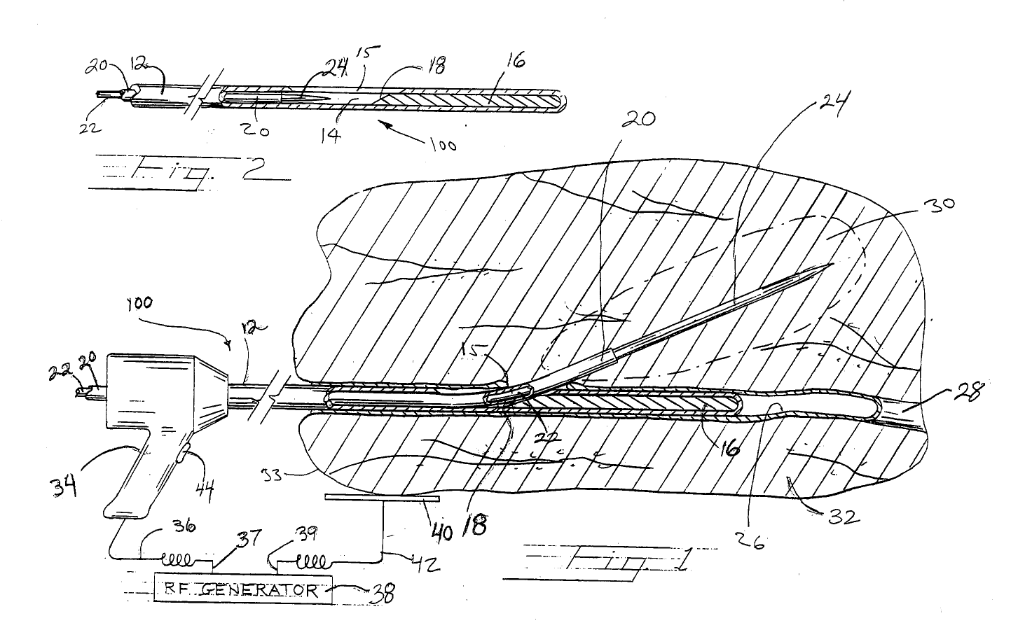

Turning now to the Drawings, FIGS. 1 and 2 depict a preferred embodiment of the medical device of the present invention positioned within a human breast for thermal treatment of breast tissue. Device 100 includes a hollow catheter 12, which defines a passageway 14 and a side port 15 in passageway 14. Catheter 12 has a closed distal end portion 16 provided with a deflector 18. Side port 15 is positioned adjacent to deflector 18. Insulating sleeve 20 is slidably disposed within passageway 14. Energy transmission line 22 is disposed within insulating sleeve 20, and terminates at its distal end in needle-shaped RF electrode 24. Electrode 24 can be passed through passageway 14 and port 15 guided by deflector 18.

As shown in FIG. 1, catheter 12 is positioned within lumen 26 of mammary duct 28. Electrode 24 is shown embedded in a suspect region 30 of breast tissue 32. Device 100 includes handle 34 at the distal end of catheter 12. Handle 34 is connected to an external lead 36, which is in operable connection with transmission line 22 and with pole 37 of an external RF generator 38. Pole 39 of generator 38 is connected to dispersive electrode 40 by lead 42. Dispersive electrode 40 is in contact with an external surface of the patient, such as the external surface of the breast 33.

In use, radiofrequency alternating current, preferably having a frequency in the range of about 500 to about 750 KHz, is supplied to electrode 24 by RF generator 38. Handle 34 includes button 44 for actuating the supply of radiofrequency energy to working electrode 24. Electrode 24 is heated by the current, which in turn, heats the suspect region of breast tissue 30 (e.g., cancerous or pre-cancerous tissue) to a temperature sufficient to inhibit cell proliferation, ablate, or destroy the tissue. After the thermal treatment, electrode 24 can be withdrawn back into passageway 14 of catheter 12 and device 100 can be removed from mammary duct 28, or device 100 can be repositioned therein for additional treatments.

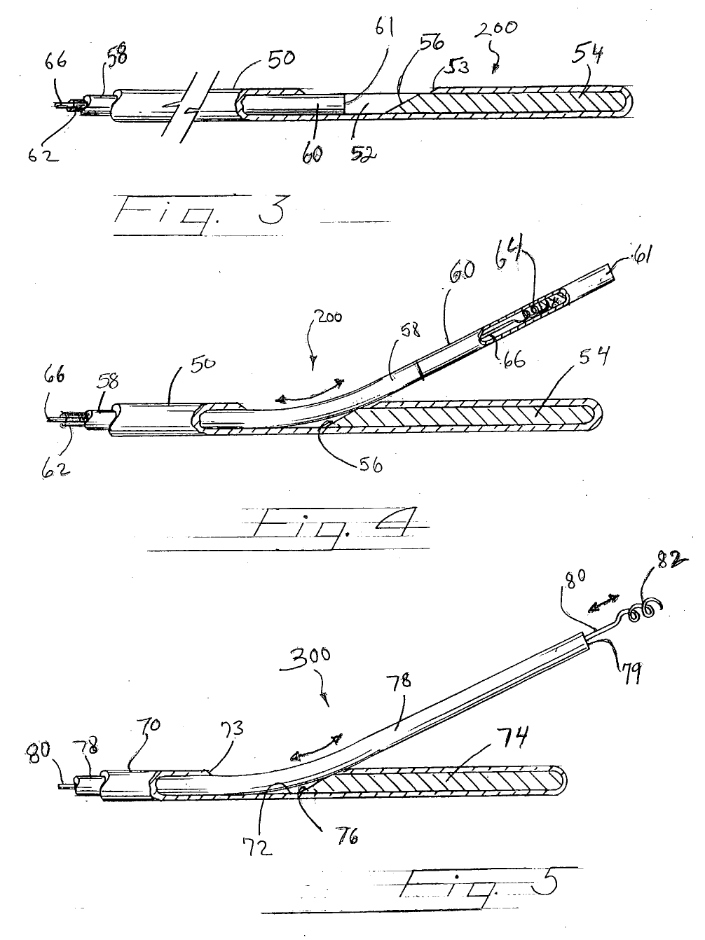

FIGS. 3 and 4 illustrate another preferred embodiment of a medical device of the present invention having a hollow ablation probe. As shown in FIG. 3, device 200 includes catheter 50 defining passageway 52 and a side port 53. Catheter 50 has a closed distal end portion 54, which contains a deflector 56 adjacent to side port 53. Insulating sleeve 58 is slidably disposed within catheter 50. Ablation probe 60, in the form of a hollow coring needle electrode, has an open distal end 61. Electromagnetic energy transmission line 62 is disposed within insulating sleeve 58 and is operably connected to probe 60.

As shown in FIG. 4, a biopsy auger 64 is moveably situated within probe 60. Auger 64 is operably connected to flexible shaft 66 within transmission line 62. When device 200 is positioned within a human breast with probe 60 penetrating a region of suspect breast tissue, auger 64 can be rotated in a corkscrew fashion so as to enter the tissue and then pulled back to remove a specimen plug of breast tissue and draw it into the interior of the conductor 62, away from probe 60. Preferably the plug is removed from device 200 by pulling the auger and associated tissue out of the device. The tissue sample can then be subjected to histological evaluation. The tissue sample can be obtained before thermal treatment, after treatment, or both, as desired.

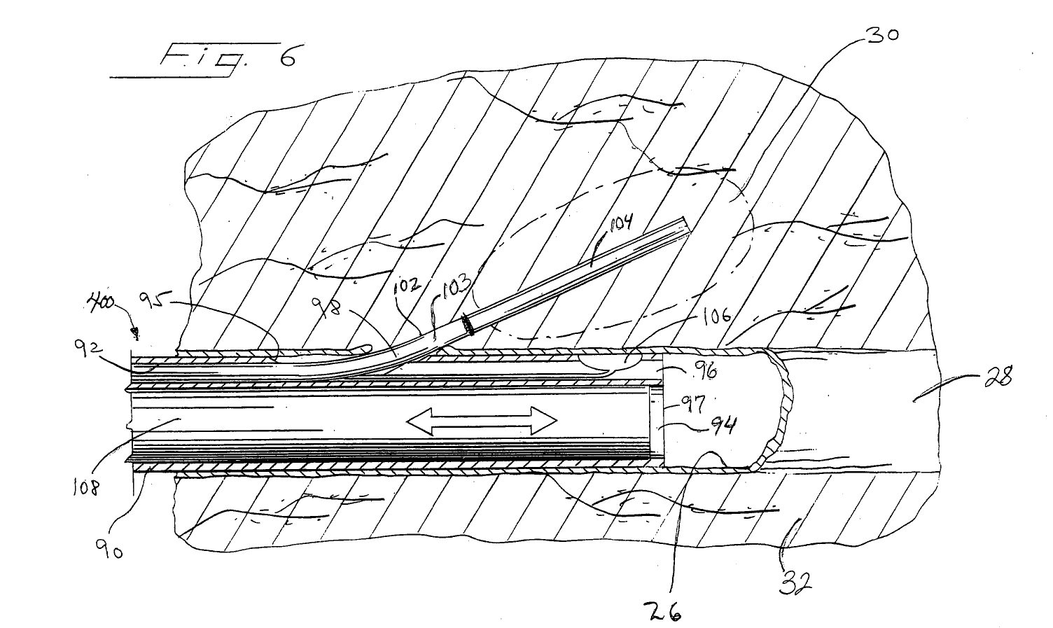

FIG. 5 illustrates an alternative embodiment of the medical device of the present invention. Device 300 includes a hollow catheter 70 defining a passageway 72 and a side port 73. Passageway 72 has a closed distal end portion 74 containing a deflector 76 adjacent to side port 73. A flexible insulating sleeve 78 is slidably disposed within catheter 70. An open distal end 79 of insulating sleeve 78 is adapted to penetrate breast tissue. Device 300 is shown with insulating sleeve extended out of side port 73, guided by deflector 76. Energy transmission line 80 is slidably disposed within insulating sleeve 78. Transmission line 80 terminates, at its distal end, in auger-shaped RF electrode 82. Auger-shaped RF electrode 82 can be rotated, in corkscrew fashion, to cut a plug of breast tissue, which can be retracted into insulating sleeve 78 for storage, or pulled out of device 300 entirely. Auger shaped RF electrode 82 then can be re-extended out from open distal end 79 of insulating sleeve 78 to heat breast tissue in the region near the electrode by applying RF energy to the electrode from an RF generator. Optionally, the tissue sample can be stored within insulating sleeve 78, and is protected from thermal damage by the insulating effect of the sleeve. Upon withdrawal of device 300 from the mammary duct, the tissue sample can be recovered for histological evaluation.

FIG. 6 illustrates a preferred dual-passageway medical device 400 of the present invention positioned for use in lumen 26 of human mammary duct 28. Device 400 includes catheter 90 defining an electrode passageway 92, a viewing passageway 94, and a side port 95 in passageway 92. Passageway 92 has a closed distal end portion 96 containing a deflector 98 adjacent to side port 95. Hollow energy transmission line 102 is coated with an insulating coating 103 and terminates in an uninsulated electrode portion 104 at its distal end. Transmission line 102 along with its associated coating 103 is slidably disposed within electrode passageway 92 of catheter 90.

Transmission line 102 is shown with electrode portion 104 extended out of side port 95 and into a suspect region of breast tissue 30, guided by deflector 98. Distal end portion 98 of electrode passageway 92 is provided with a thermal sensor 106 that is adapted for connection to a temperature indicator or to a feedback loop on an RF generator to control the temperature of the tissue during treatment. Alternatively, the thermal sensor can be located at the tip of electrode portion 104. Preferably, the thermal sensor is insulated from the electrode by a polymeric coating, such as a polyamide coating. Optionally, a biopsy auger can be disposed within the hollow transmission line 102 to retrieve tissue samples before and/or after treatment, as described above.

Viewing passageway 94 has an open distal end 97. An fiber optic viewing scope 108 is slidably disposed within viewing passageway 94, and is adapted for transmitting images from the open distal end 97 of second passageway 94 to an external viewing device, such as a video monitor (not shown).

In operation, device 400 functions substantially as described for devices 100, 200 and 300 above, with the exception that the temperature of the tissue adjacent to the distal end portion 96 of electrode passageway 92 can be monitored by the thermal sensor during the heating of the suspect breast tissue 30 and the internal surface of the mammary duct 28 can be viewed through viewing scope 108.

FIG. 7 depicts a particularly preferred embodiment of the medical device of the present invention. Device 500 includes a catheter 150 defining a passageway 152 and having an open distal end 154. An insulating sleeve 156 is slidably disposed within passageway 152. Hollow energy transmission line 158 is disposed within sleeve 156 and terminates at its distal end in hollow electrode 160. Working passageway 162 with an open, beveled distal end 164 for penetrating breast tissue is defined within hollow electrode 160. Fiber optic viewing scope 166 is disposed within working passageway 162. Viewing scope 166 is adapted to transmit images from the open distal end 164 of electrode 160 to an external viewing apparatus such as a video monitor and the like.

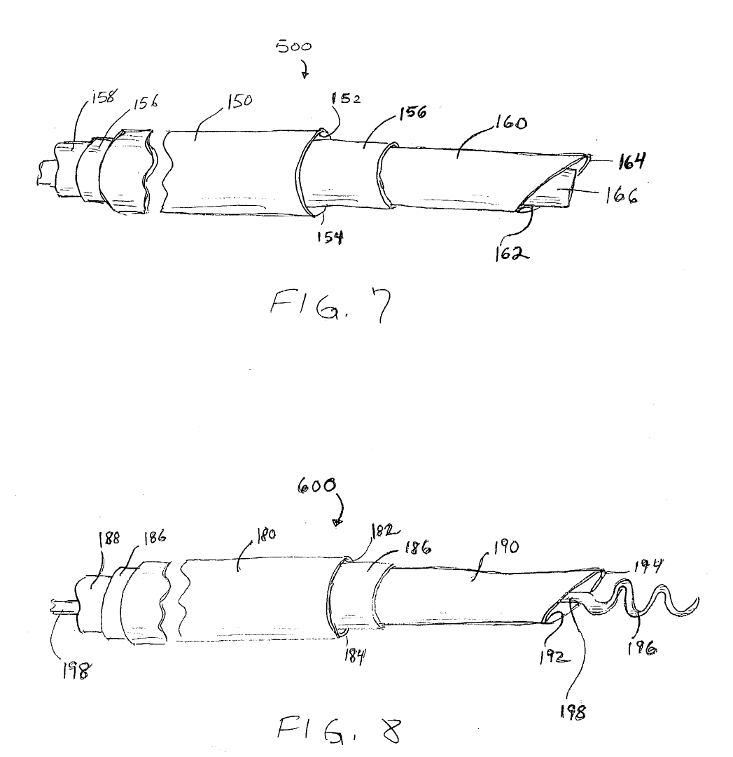

FIG. 8 depicts an alternative preferred configuration of the medical device of FIG. 7. Device 600 includes a catheter 180 defining a passageway 182 and having an open distal end 184. An insulating sleeve 186 is slidably disposed within passageway 182. Hollow energy transmission line 188 is disposed within sleeve 186 and terminates at its distal end in hollow electrode 190. Electrode 190 defines working passageway 192 and has an open, beveled distal end 194 for penetrating breast tissue. Biopsy auger 196 and its attached control rod 198 are slidably disposed within working passageway 192. Auger 196 is adapted to penetrate breast tissue and retrieve a tissue sample therefrom. Alternatively, auger 196 can serve as the electrode, if it is conductive.

Optionally, viewing scope 166 of device 500 and/or auger 196 of device 600 can be replaced with a thermal probe before RF energy is supplied to the electrode. The probe can be extended into the tissue and used to monitor or control the tissue temperature during treatment. The probe can be connected to a temperature monitor or to a feedback loop for controlling the current applied to the electrode. Preferably the thermal probe is a thermistor probe.

The devices of the present invention can be provided in the form of a kit, which preferably includes, in addition to the device of the present invention as described above, instructional indicia, such as printed instructions, diagrams, video tape, interactive CD-ROM or DVD-ROM or other electronic media, and the like materials, which describe the use of the device to treat a suspect region of breast tissue as described above. The kit can also include accessories such as optical scopes, augers, a selection of different ablation probes, a dispersive electrode, and the like, and combinations of the foregoing. The instructional indicia preferably describe the use of the accessories in conjunction with the device to thermally treat breast tissue, retrieve biopsy samples, and the like.

The foregoing description is to be taken as illustrative, but not limiting. Still other variants within the spirit and scope of the present invention will readily present themselves to those skilled in the art.

We claim:

1. A medical device suitable for delivery of electromagnetic energy to breast tissue, and comprising;
(a) a hollow catheter sized to fit within a mammary duct of a patient and defining a passageway having a distal end portion and an open proximal end;
(b) an elongate insulating sleeve slidably disposed within the passageway;
(c) an electromagnetic energy transmission line within the insulating sleeve adapted for connection to an electromagnetic energy generator; and
(d) an elongate, flexible ablation probe operably coupled to the electromagnetic transmission line at its distal end and configured for generating an electromagnetic field sufficient to cause tissue ablation, the probe being adapted for penetration of breast tissue.

2. The medical device of claim 1 wherein the ablation probe is a microwave antenna.

3. The medical device of claim 1 wherein the ablation probe is a radiofrequency electrode.

4. The medical device of claim 3 wherein the radiofrequency electrode is hollow and open ended.

5. The medical device of claim 4 further comprising a fiber optic viewing probe within the radiofrequency electrode.

6. The medical device of claim 4 wherein the hollow radiofrequency electrode is in the form of a coring needle.

7. The medical device of claim 4 further including a removable auger within the radiofrequency electrode, such that when the electrode is positioned within a region of breast tissue in need of thermal treatment, the auger is adapted for collection of a breast tissue sample.

8. The device of claim 1 including a handle spaced from the distal end portion of the catheter and affixed thereto.

9. The device of claim 8 wherein the catheter has an open distal end and the handle includes a mechanism for extending the ablation probe through the open distal end of the catheter.

10. The device of claim 1 wherein the electrode includes a thermal sensor at the distal end thereof.

11. The device of claim 10 wherein the thermal sensor is a thermistor.

12. The device of claim 11 wherein the thermistor is adapted for connection to a temperature indicator.

13. The device of claim 11 wherein the thermistor is part of a feedback loop for regulation of the electromagnetic energy generator.

14. The device of claim 1 further including a handle at the proximal end of the catheter.

15. The device of claim 14 wherein the handle includes a mechanism for manipulating the ablation probe within the passageway.

16. A medical device suitable for delivering radiofrequency energy to breast tissue, and comprising:
(a) a hollow catheter defining a passageway having a closed distal end portion, an open proximal end, and a side port adjacent to the distal end portion;
(b) a deflector within the distal end portion of the passageway;
(c) an elongate insulating sleeve slidably disposed within the passageway; and
(d) an electromagnetic energy transmission line within the insulating sleeve, the proximal end of the transmission line being adapted for connection to a radiofrequency generator, the distal end of the transmission line terminating in a radiofrequency electrode adapted for penetration of breast tissue;
wherein the catheter is sized to fit within the lumen of a human mammary duct; such that the catheter can be positioned within a mammary duct, and the electrode is adapted to pass through the passageway and the side port guided by the deflector.

17. The device of claim 16 wherein the transmission line is slidably disposed within the insulating sleeve.

18. The device of claim 17 wherein the insulating sleeve is adapted for penetrating breast tissue.

19. The device of claim 16 wherein the electrode is in the form of a needle.

20. The device of claim 16 wherein the electrode is in the form of an auger.

21. The device of claim 16 wherein the electrode is a hollow coring needle, and an auger is slidably situated within the coring needle.

22. The device of claim 16 including a handle at the proximal end of the catheter.

23. The device of claim 20 wherein the handle includes a mechanism for extending the electrode through the side port of the catheter.

24. The device of claim 14 wherein the electrode includes a thermal sensor at the distal end thereof.

25. The device of claim 24 wherein the thermal sensor is a thermistor.

26. The device of claim 25 wherein the thermistor is adapted for connection to a temperature indicator.

27. The device of claim 25 wherein the thermistor is part of a feedback loop for regulating the electromagnetic energy generator.

28. A medical device suitable for delivering electromagnetic energy to breast tissue, and comprising:
(a) a hollow catheter defining a probe passageway having an open distal end portion, an open proximal end, and further defining a viewing passageway having an open proximal end and an open distal end;
(b) an elongate insulating sleeve slidably disposed within the probe passageway; and
(c) an electromagnetic energy transmission line within the insulating sleeve, the proximal end of the transmission line being adapted for connection to an electromagnetic energy generator, the distal end of the transmission line terminating in an ablation probe adapted for penetration of breast tissue;
wherein the catheter is sized to fit within the lumen of a human mammary duct; such that the catheter can be positioned within a mammary duct, and the ablation probe is adapted to pass through the open distal end of the probe passageway.

29. The device of claim 28 wherein the transmission line is slidably disposed within the insulating sleeve.

30. The device of claim 29 wherein the insulating sleeve is adapted for penetrating breast tissue.

31. The device of claim 28 wherein the ablation probe is in the form of an auger.

32. The device of claim 28 wherein the ablation probe is in the form of a needle.

33. The device of claim 28 wherein the ablation probe is a hollow coring needle and an auger is slidably disposed within the coring needle.

34. The device of claim 28 including a handle at the proximal end of the catheter.

35. The device of claim 34 wherein the handle includes a mechanism for extending the ablation probe through the probe passageway.

36. The device of claim 28 wherein the ablation probe includes a thermal sensor at its distal end.

37. The device of claim 36 wherein the thermal sensor is a thermistor.

38. The device of claim 37 wherein the thermistor is adapted for connection to a temperature indicator.

39. The device of claim 37 wherein the thermistor part of a feedback loop for regulating the electromagnetic energy generator.

40. The device of claim 28 further comprising a fiber optic viewing scope within the viewing passageway of the catheter adapted for transmitting images from the open distal end of the viewing passageway to an external viewing apparatus.

41. The device of claim 40 wherein the fiber optic viewing scope is slidably disposed within the viewing passageway.

42. A kit comprising a device of claim 1 and instructional indicia for using the device in combination with an electromagnetic energy generator to thermally treat breast tissue.

43. The kit of claim 42 further comprising a dispersive electrode adapted for connection to a radiofrequency generator.

44. A kit of claim 43 wherein the ablation probe is a radiofrequency electrode and instructional indicia for using the device in combination with a radiofrequency generator to thermally treat breast tissue.

45. A method of thermally treating breast tissue in a human patient with a device of claim 1, and comprising the steps of:
(a) positioning the distal end portion of the catheter within the lumen of a human mammary duct adjacent to a region of breast tissue in need of thermal treatment;
(b) passing the ablation probe through the wall of the mammary duct and into the region of breast tissue in need of thermal treatment;
(d) placing the ablation probe in operative relationship with a electromagnetic energy generator; and
(e) supplying an effective amount of electromagnetic energy from the generator to the ablation probe so as to thermally treat the region of breast tissue in need of treatment.

46. The method of claim 45 wherein the ablation probe is hollow and open ended.

47. The method of claim 46 wherein device includes a removable auger within the ablation probe adapted for collecting a breast tissue sample.

48. The method of claim 47 further including the step of collecting a tissue sample from the region of breast tissue in need of thermal treatment with the auger prior to supplying electromagnetic energy to the ablation probe.

49. The method of claim 46 wherein the device includes a fiber optic viewing scope adapted for transmitting images of the mammary duct from the distal end of the catheter to an external viewing apparatus.

50. The method of claim 45 wherein the electromagnetic energy is radiofrequency energy.

51. The method of claim 45 wherein the electromagnetic energy is microwave energy.
